(19) United States
(12) Patent Application Publication
(10) Pub. No.: US 2025/0368602 A1
(43) Pub. Date: Dec. 4, 2025

(54) PROCESS FOR PREPARING CYANO SUBSTITUTED ANTHRANILIC DIAMIDES AND INTERMEDIATES THEREOF

(71) Applicant: PI INDUSTRIES LTD., Udaipur-Rajasthan (IN)

(72) Inventors: Nitin MALVIYA, Rajasthan (IN); Pankaj NILKANTH, Rajasthan (IN); Sachin Sahebrao MUSMADE, Rajasthan (IN); Alexander G.M. KLAUSENER, Pulheim (DE)

(73) Assignee: PI INDUSTRIES LTD., Udaipur-Rajasthan (IN)

(21) Appl. No.: 19/300,047

(22) Filed: Aug. 14, 2025

Related U.S. Application Data
(63) Continuation-in-part of application No. 17/310,673, filed on Aug. 17, 2021, now Pat. No. 12,410,158.

(30) Foreign Application Priority Data
Feb. 18, 2019 (IN) 201911006381

(51) Int. Cl. C07C 231/14; C07D 401/14

(52) U.S. Cl. CPC C07C 231/14; C07D 401/14; C07C 2527/122

(57) ABSTRACT

The present invention relates to a novel process for preparing a compound of Formula I and the intermediates thereof,

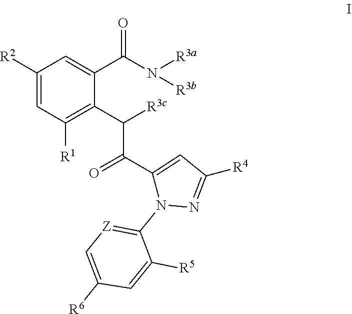

wherein, R¹, R², R^{3a}, R^{3b}, R^{3c}, R⁴, R⁵, R⁶, R⁷ and Z are as defined in the description.

This application is a continuation-in-part of U.S. application Ser. No. 17/310,673 filed Aug. 17, 2021 as a National Stage Entry of International Application No. PCT/IB2020/051227, filed Feb. 14, 2020, and entitled “A PROCESS FOR PREPARING ANTHRANILIC DIAMIDES AND INTERMEDIATES THEREOF;” which claims priority to Indian Application No. 201911006381, filed Feb. 18, 2019, and entitled “A PROCESS FOR PREPARING ANTHRANILIC DIAMIDES AND INTERMEDIATES THEREOF,” the contents of which are incorporated by reference herein.

### FIELD OF THE INVENTION

The present invention relates to a process for preparing cyano substituted anthranilic diamides involving the conversion of isatins into isatoic anhydrides. The present invention also relates to a process for preparing cyano substituted anthranilic amides useful in the process for preparing cyano substituted anthranilic diamides.

### BACKGROUND OF THE INVENTION

WO2003015518, WO2003015519, WO2004067528, WO2005077934 and WO20100069502 disclose the use of anthranilic diamides for controlling invertebrate pests such as arthropods.

Several patent documents, for example WO2004011447, WO2004111030, WO2006062978, WO2008010897 and WO2012103436 disclose processes for preparing anthranilic diamides and suitable intermediates.

Further, WO2020170092 disclosed the process for the preparation of anthranilic diamides and its intermediates such as anthranilic amides. The process disclosed in this invention describes the preparation of halogen substituted anthranilic diamides and anthranilic amides compounds however, there is no method disclosed how to prepare cyano substituted anthranilic diamides and anthranilic amide compounds.

The present invention is further improvement/modification of process described in WO2020170092 to obtain the cyano substituted anthranilic diamides and anthranilic amide compounds.

### OBJECT AND SUMMARY OF THE INVENTION

It is an objective of the present invention to provide an industrially amenable and convenient process for the preparation of cyano substituted anthranilic diamides of Formula I.

The present invention provides a solution to this objective by providing an improved process that allows the preparation of cyano substituted anthranilic diamides, overcoming at least one of the shortcomings of the processes described in the prior art.

The said objective was achieved according to the present invention by providing an improved process for preparing a compound of Formula I,

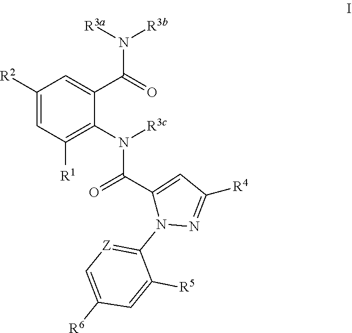

wherein,
R¹ is CH₃, Br or Cl;
R² is CN;
R^{3a} and R^{3b} are independently H, C₁-C₄ alkyl or C₃-C₆ cycloalkyl-C₁-C₄ alkyl;
R^{3c} is H or C₁-C₄ alkyl;
R⁴ is Cl, Br, CF₃, OCF₂H, OCH₂CF₃,

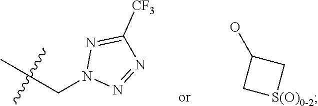

R⁵ is F, Cl or Br;
R⁶ is H, F or Cl;
Z is CR⁷ or N; and
R⁷ is H, F, Cl or Br.

The process according to this invention comprises the step of obtaining a dione of Formula II from an aniline of Formula III and chloral hydrate of Formula IV, and converting the dione of Formula II into an isatoic anhydride of Formula V in a single step. The conversion of the dione of Formula II into an isatoic anhydride of Formula V is novel and inventive as both the oxidation and the halogenation reactions are carried out in a single step.

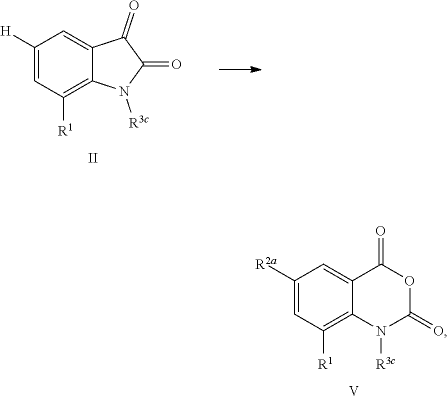

wherein, R^{2a} is F, Cl, Br or I; R¹ and R^{3c} are as defined hereinabove.

The isatoic anhydride of Formula V and an amine of Formula VI are reacted to obtain a compound of Formula VII-A which was converted to a compound of Formula VII,

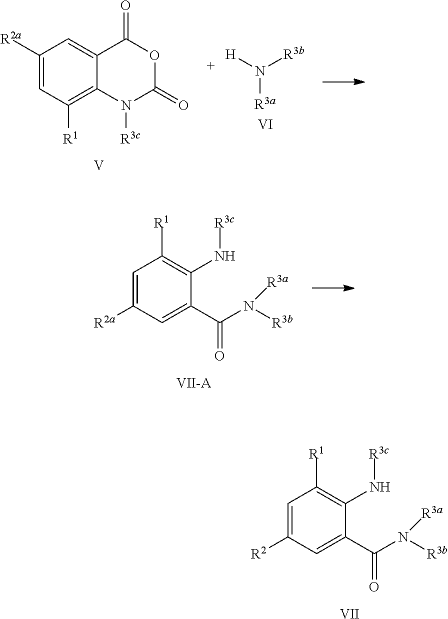

The compound of Formula VII was further reacted with a compound of Formula VIII to obtain the compound of Formula I,

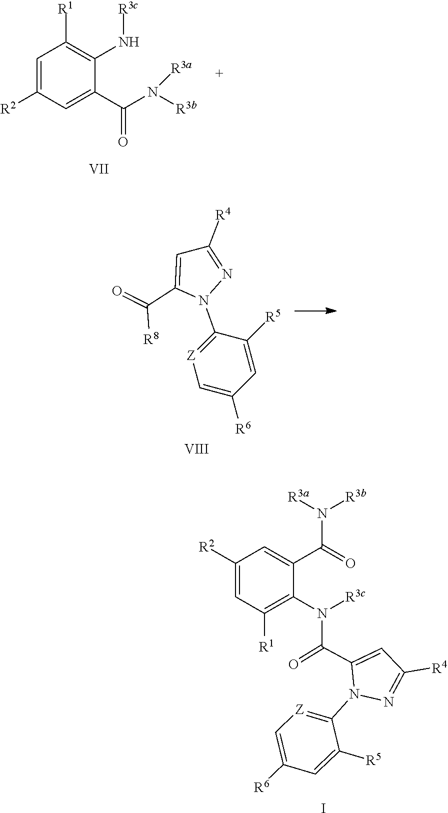

wherein, R¹, R², R^{3a}, R^{3b}, R^{3c}, R⁴, R⁵, R⁶ and Z are as defined for Formula I; and R⁸ is OH, C, X or O—C₁-C₄ alkyl.

In another embodiment, the present invention provides an improved process for the preparation of compound of Formula (VII)

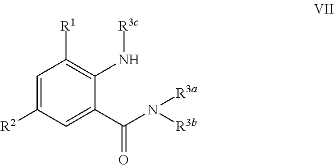

wherein,
R¹ is CH₃, Br or Cl;
R² is CN;
R^{3a} and R^{3b} are independently H, C₁-C₄ alkyl or C₃-C₆ cycloalkyl-C₁-C₄ alkyl;
R^{3c} is H or C₁-C₄ alkyl;
   comprising the steps of:
a) oxidizing and halogenating the dione of Formula II simultaneously into an isatoic anhydride of Formula V using at least one oxidizing reagent and at least one halogenating reagent optionally in the presence of at least one suitable acid;

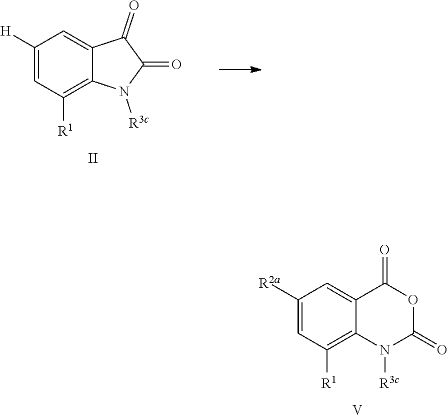

wherein, R^{2a} is F, Cl, Br, or I; R¹ and R^{3c} are as defined herein before,
b) reacting the compound of Formula V with an amine of Formula VI to obtain a compound of Formula VII-A in the presence of at least one suitable reagent;

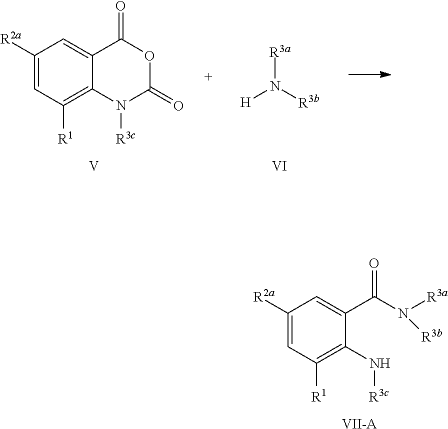

wherein, R^{2a} is F, Cl, Br, or I; R¹, R^{3a}, R^{3b} and R^{3c} are as defined herein before, and
c) reacting the compound of Formula VII-A with cyano source to obtain a compound of Formula VII in the presence of a suitable solvent;

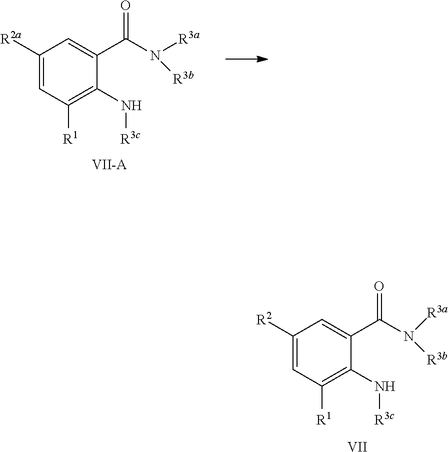

wherein, R^{2a} is F, Cl, Br, or I; R¹, R², R^{3a}, R^{3b} and R^{3c} are as defined herein before.

### DETAILED DESCRIPTION OF THE INVENTION

### General Definitions

The definitions provided herein for the terminologies used in the present disclosure are for illustrative purposes only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Carbon-based radical refers to a monovalent molecular component comprising a carbon atom that connects the radical to the remainder of the chemical structure through a single bond. Carbon-based radicals can optionally comprise saturated, unsaturated and aromatic groups, chains, rings and ring systems, and heteroatoms. Although carbon-based radicals are not subject to any particular limit in size, in the context of the present invention they typically comprise 1 to 16 carbon atoms and 0 to 3 heteroatoms. Of note are carbon-based radicals selected from C₁-C₆ alkyl, C₁-C₆ haloalkyl and phenyl optionally substituted with 1-3 substituents selected from C₁-C₃ alkyl, halogen and nitro.

The meaning of various terms used in the description shall now be illustrated.

The term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Representative examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “cycloalkyl” means alkyl closed to form a ring. Representative examples include but are not limited to cyclopropyl, cyclobutyl, cyclopentyl and cyclohexyl. This definition also applies to cycloalkyl as a part of a composite substituent, for example cycloalkylalkyl etc., unless specifically defined elsewhere.

Accordingly, in one embodiment, the present invention provides a method for the preparation of compound of Formula (VII)

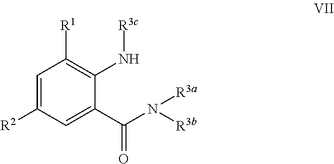

wherein,
R¹ is CH₃, Br or Cl;
R² is CN;
R^{3a} and R^{3b} are independently H, C₁-C₄ alkyl or C₃-C₆ cycloalkyl-C₁-C₄ alkyl;
R^{3c} is H or C₁-C₄ alkyl;
   comprising the steps of:
a) oxidizing and halogenating the dione of Formula II simultaneously into an isatoic anhydride of Formula V using at least one oxidizing reagent and at least one halogenating reagent optionally in the presence of at least one suitable acid;

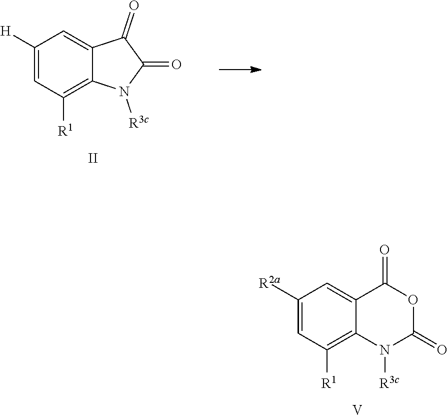

wherein, R^{2a} is F, Cl, Br, or I; R¹ and R^{3c} are as defined herein before,
b) reacting the compound of Formula V with an amine of Formula VI to obtain a compound of Formula VII-A in the presence of at least one suitable reagent;

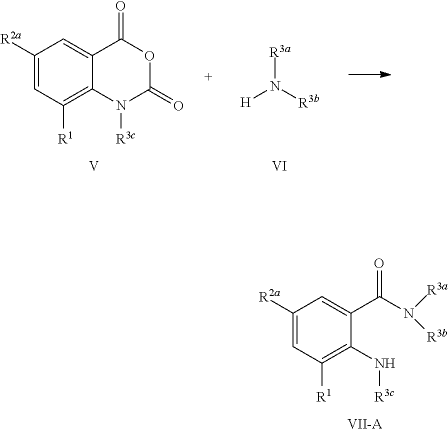

wherein, R^{2a} is F, Cl, Br, or I; R¹, R^{3a}, R^{3b} and R^{3c} are as defined herein before, and
c) reacting the compound of Formula VII-A with cyano source to obtain a compound of Formula VII in the presence of suitable solvent;

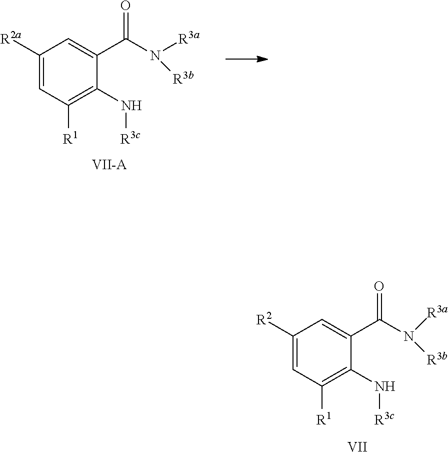

wherein, R^{2a} is F, Cl, Br, or I; R¹, R², R^{3a}, R^{3b} and R^{3c} are as defined herein before.

In another embodiment, the present invention relates to a process for preparing a compound of Formula I,

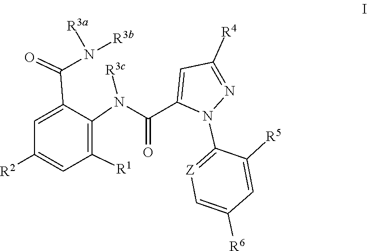

wherein,
R¹ is CH₃, Br or Cl;
R² is CN;
R^{3a} and R^{3b} are independently H, C₁-C₄ alkyl or C₃-C₆ cycloalkyl-C₁-C₄ alkyl;
R^{3c} is H or C₁-C₄ alkyl;
R⁴ is Cl, Br, CF₃, OCF₂H, OCH₂CF₃, or

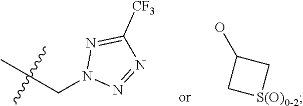

R⁵ is F, Cl or Br;
R⁶ is H, F or Cl;
Z is CR⁷ or N; and
R⁷ is H, F, Cl or Br.

The process of the present invention is described herein after.

Aniline of Formula III and chloral hydrate of Formula IV were reacted to obtain dione of Formula II

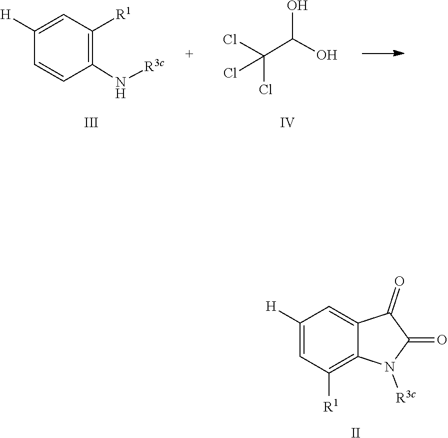

wherein, R¹ and R^{3c} are as defined herein above.

In one embodiment, the compound of Formula III and chloral hydrate of Formula IV are reacted in the presence of one or more suitable reagent including but not limiting to hydrochloric acid, sulfuric acid, acetic acid, trifluoroacetic acid, nitric acid and sodium sulphate in one or more solvent/s at a temperature ranging from 25° C. to 100° C., followed by stirring with mineral acid including but not limiting to sulfuric acid, hydrochloric acid and nitric acid at 0° C. to 45° C. to obtain the dione of Formula II.

In another embodiment, the oxime of Formula IIIa is formed by reacting the compound of Formula III and chloral hydrate of Formula IV or hydroxylamine in the presence of one or more suitable reagent including but not limiting to hydrochloric acid, sulfuric acid, acetic acid trifluoroacetic acid, nitric acid and sodium sulphate and one or more solvent at a temperature ranging from 15° C. to 150° C.,

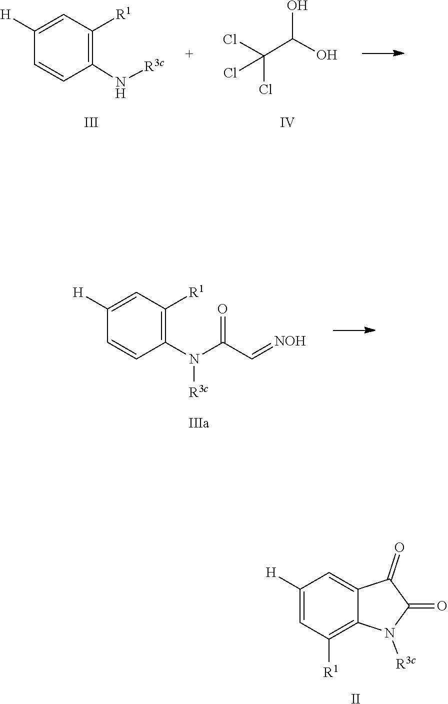

wherein, R¹ and R^{3c} are as defined herein above.

The solvent useful in this step includes but is not limited to aliphatic hydrocarbons such as hexane, heptane, octane, nonane, decane, dodecane and the like; alicyclic hydrocarbons such as cycloalkanes: cyclopropane, cyclobutane, cyclopentane, cyclohexane, cycloheptane, cyclooctane, and the like; aromatic hydrocarbons such as toluene, xylene, mesitylene, benzene and the like; ethers such as diisopropyl ether, t-butyl methyl ether, tetrahydrofuran, 2-methyl tetrahydrofuran, methyl alcohol, ethyl alcohol, acetone, dioxane, monoglyme, diglyme, methoxy-methane, methoxy-ethane, ethoxy-ethane, di-methoxyethane, di-ethoxyethane and the like; halogenated hydrocarbons such as dichloromethane, chloroform, dichloroethane and like; ethers, polar aprotic solvents such as N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, 1,3-dimethyl-2-imidazolidinone and the like; and water.

The oxime of Formula IIIa is then converted into the compound of Formula II by using mineral acid including but not limiting to sulfuric acid, hydrochloric acid and nitric acid and stirring within a temperature ranging from 0° C. to 150° C.

The dione of Formula II is converted into an isatoic anhydride of Formula V using a suitable halogenating reagent, one or more suitable oxidizing reagent/s and one or more suitable solvent at a temperature ranging from 0° C. to 250° C.,

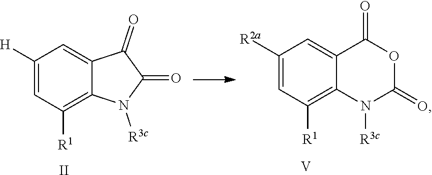

wherein, R^{2a} is F, Cl, Br or I; R¹ and R^{3c} are as defined herein above.

The halogenating reagent useful for converting the dione of Formula II into isatoic anhydride of Formula V includes but is not limited to HX, NaX, KX, CuX₂, MgX₂, CsX, ZnX₂, SOCl₂, SO₂Cl₂, COCl₂, X₂, C(═O)(OCl₃)₂, t-BuOCl, NaOCl, Chloramine-T, N-halosuccinamides, POX₃, PX₃, PX₅ or metal halides; wherein X is Cl, Br, I or F.

The oxidizing reagents useful for converting the dione of Formula II into the isatoic anhydride of Formula V includes but is not limited to hydrogen peroxide, t-butyl-hydroperoxide, tungstic peroxide, m-chloroperbenzoic acid, benzoyl peroxide, hypohalous acid, ceric ammonium nitrate, hypoceric ammonium nitrate, oxone, periodic acid, hydrogen peroxide urea-adduct, sodium perborate, pyridinium chlorochromate and dimethyl sulfoxide.

The acid reagents which is optionally used for converting the dione of Formula II into the isatoic anhydride of Formula V includes but is not limited formic acid, acetic acid, triflic acid, benzoic acid, m-chlorobenzoic acid, butyric acid, propionic acid, glycolic acid, trifluoroacetic acid, para-toluene sulfonic acid, methane sulfonic acid, butyric acid, citric acid, oxalic acid, malonic acid, maleic acid, gallic acid, tartaric acid, ascorbic acid, hydrochloric acid, hydroiodic acid, sulphuric acid, nitric acid, phosphoric acid, and perchloric acid;

The solvent useful for converting the dione of Formula II into the isatoic anhydride of Formula V includes but is not limited to an organic acid selected from the group consisting of formic acid, acetic acid, triflic acid, butyric acid, propionic acid, carbonic acid, glycolic acid, and trifluoroacetic acid.

Alternatively, the solvents useful for converting the dione of Formula II into the isatoic anhydride of Formula V includes but is not limited to a mixture of said organic acid/s with one or more solvent/s selected from the group comprising of aliphatic hydrocarbons such as hexane, heptane, octane, nonane, decane, dodecane and the like; alicyclic hydrocarbons such as cycloalkanes: cyclopropane, cyclobutane, cyclopentane, cyclohexane, cycloheptane, cyclooctane, and the like; aromatic hydrocarbons such as toluene, xylene, mesitylene, benzene and the like; ethers such as diisopropyl ether, t-butyl methyl ether, tetrahydrofuran, 2-methyl tetrahydrofuran, methyl alcohol, ethyl alcohol, acetone, dioxane, monoglyme, diglyme, methoxy-methane, methoxy-ethane, ethoxy-ethane, di-methoxyethane, di-ethoxyethane and the like; halogenated hydrocarbons such as dichloromethane, chloroform, dichloroethane and the like; polar aprotic solvents such as N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, 1,3-dimethyl-2-imidazolidinone, acetic acid, hydrochloric acid, hydrobromic acid, sulfuric acid and the like; and water.

In one embodiment, the step of converting the dione of Formula II into the isatoic anhydride of Formula V is carried out by mixing i) a mixture of the halogenating reagent and the oxidizing reagent, and ii) a mixture of the dione of Formula II and the solvent, at a temperature ranging from 10 to 50° C. and then by heating at a temperature ranging from 15-150° C.

In another embodiment, the step of converting the dione of Formula II into the isatoic of Formula V is carried out by mixing i) the halogenating reagent, and ii) the oxidizing reagent separately in either sequence with iii) a mixture of the dione of Formula II and the solvent, at a temperature ranging from 10 to 50° C., followed by heating at a temperature ranging from 15-150° C.

In one embodiment of the present invention, step-(a) is carried out in acetic acid as solvent in the proportion of 2 to 10 w/w volumes with respect to compound of formula (II).

In one embodiment of the present invention, halogenation and oxidation in step-(a) is carried out in the presence of HX/H₂O₂ reagent in the proportion of 1 to 10 molar equivalents with respect to compound of formula (II).

In one embodiment of the present invention, sulfuric acid used in step-(a) is in the proportion of 0.01 to 0.5 molar equivalents with respect to compound of formula (II); preferably 0.01 to 0.1 molar equivalents.

In the next step, the isatoic anhydride of Formula V and an amine of Formula VI are reacted to obtain a compound of Formula VII-A,

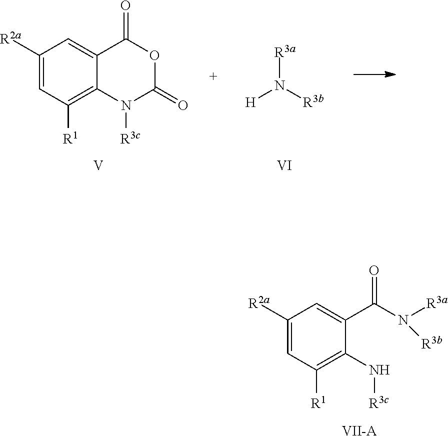

wherein, R^{2a} is F, Cl, Br or I; R¹, R², R^{3a}, R^{3b} and R^{3c} are as defined herein before.

The amine of Formula VI may be used in aqueous form or a gaseous form. For example, when R^{3a} is methyl, then methyl amine is used for the preparation of compound of Formula VII, wherein R^{3a} is methyl; in this case methyl amine may be used in gaseous form or may be used as a solution in water or one or more solvent/s.

The solvents being useful for this reaction are preferably selected from the group comprising of aliphatic hydrocarbons such as hexane, heptane, octane, nonane, decane, dodecane and the like; alicyclic hydrocarbons such as cycloalkanes: cyclopropane, cyclobutane, cyclopentane, cyclohexane, cycloheptane, cyclooctane, and the like; aromatic hydrocarbons such as toluene, xylene, mesitylene, benzene and the like; ethers such as diisopropyl ether, t-butyl methyl ether, tetrahydrofuran, 2-methyl tetrahydrofuran, methyl alcohol, ethyl alcohol, acetone, dioxane, monoglyme, diglyme, methoxy-methane, methoxy-ethane, ethoxy-ethane, di-methoxyethane, di-ethoxyethane and the like; halogenated hydrocarbons such as dichloromethane, chloroform, dichloroethane and like; ethers, polar aprotic solvents such as N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, 1,3-dimethyl-2-imidazolidinone and the like; and water.

The conversion of the isatoic anhydride of Formula V and the amine of Formula VI may further require the presence of a suitable reagent selected from the group consisting of formic acid, acetic acid, triflic acid, benzoic acid, m-chlorobenzoic acid, butyric acid, propionic acid, glycolic acid, trifluoroacetic acid, para-toluene sulfonic acid, methane sulfonic acid, butyric acid, citric acid, oxalic acid, malonic acid, maleic acid, acid, tartaric acid, ascorbic acid, hydrochloric acid, sulphuric acid, nitric acid, phosphoric acid, perchloric acid, boronic acids, amberlysts, aluminum chloride, zinc chloride, boron trifluoro ether, zinc oxide, sodium carbonate, potassium carbonate, sodium bicarbonate, potassium bicarbonate, pyridine, N-methyl-2-pyrrolidone, and N,N-dimethylmethanamide;

The temperature conditions employed for the conversion of the isatoic anhydride of Formula V and the amine of Formula VI range from 0° C. to 150° C., depending on the solvent used and the amine reactant employed.

In one embodiment, the compound of Formula V is isolated.

In another embodiment, the compound of Formula V is not isolated.

In one embodiment, the isatoic anhydride of Formula V, wherein in R^{2a} is F, Cl, Br or I, can be converted into a compound of Formula VII, wherein R² is CN.

In another embodiment the compound of formula VII-A is converted to compound of formula VII using the cyano sources, in the presence of suitable solvent and optionally in the presence of ligand and catalyst,

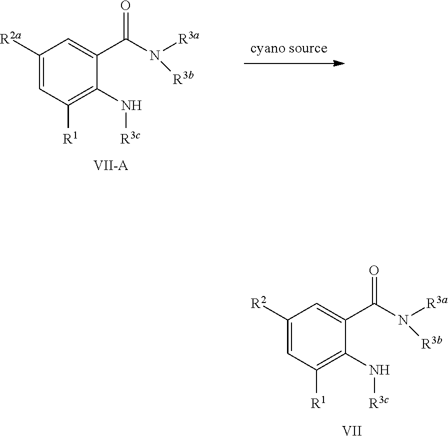

wherein, R^{2a} is F, Cl, Br, or I; R¹, R², R^{3a}, R^{3b} and R^{3c} are as defined herein before.

In one embodiment, the cyanation reactions or cyanation mentioned in step-c of the present invention is carried out in the presence of metal cyanides as a CN source.

In one embodiment, to obtain the compound of formula VII from compound of formula VIIA, the source of CN ions in carrying out the cyanation reactions or cyanation mentioned in step-c of the present invention is selected from metal cyanide particularly, sodium cyanide (NaCN), potassium cyanide (KCN), copper(I) cyanide (CuCN), zinc(II) cyanide (Zn(CN)₂), sodium ferrocyanide (Na₄Fe(CN)₆), Potassium hexacyanoferrate (III) (K₃[Fe(CN)₆]), Potassium hexacyanoferrate (II) (K₄[Fe(CN)₆]). In one embodiment, to obtain the compound of formula VII from compound of formula VIIA, the source of CN ions in carrying out the cyanation reactions or cyanation mentioned in step-c of the present invention are selected from metal cyanide is Potassium hexacyanoferrate (II) (K₄[Fe(CN)₆]).

In one embodiment, the cyanation reactions or cyanation mentioned in step-c the present invention is carried out in the presence of ligand and/or catalyst.

The ligand used in carrying out the cyanation reactions or cyanation mentioned in step-c of the present invention is selected from 1,2-dimethylethylenediamine (DMEDA), imidazole, 1-butyl-1H-imidazole, 2-picoline, 3-picoline, 2,6-lutidine, 1-methyl pyrolidine, or pyrolidine.

The ligand used in carrying out the cyanation reactions or cyanation mentioned in step-c of the present invention is imidazole.

The catalyst used in carrying out the cyanation reactions or cyanation mentioned in step-c of the present invention is selected from copper salts such as copper (I) iodide (CuI), copper (I) chloride, copper (I) bromide, copper acetate.

The catalyst used in carrying out the cyanation reactions or cyanation mentioned in step-c of the present invention is selected from copper salts preferably copper (I) iodide (CuI).

The temperature at which the cyanation reaction or cyanation mentioned in step-c is carried out is in the range of 30° C. to 200° C.

The solvents useful in carrying out the cyanation reaction or cyanation mentioned in step c of the present invention are selected from N,N-dimethylmethanamide, N,N-dimethylacetamide, N-methyl-2-pyrrolidone, 1,3-dimethyl-3,4,5,6-tetrahydro-2 (1H)-pyrimidinone, hexamethylphosphoramidem, or 1,3-dimethyl-2-imidazolidinone; more preferably N,N-dimethylformamide, N-methyl-2-pyrrolidone; preferably amide solvents selected from N,N-dimethylmethanamide, N,N-dimethylacetamide, or N-methyl-2-pyrrolidone.

The solvents useful in carrying out the cyanation reaction or cyanation mentioned in step c of the present invention is preferably selected from N,N-dimethylformamide, N,N-dimethylacetamide, or N-methyl-2-pyrrolidone.

In one embodiment of the present invention, copper (I) iodide used in step-(c) is in the proportion of 0.08 to 0.01 molar equivalents with respect to compound of formula (VII-A); preferably 0.05 to 0.03 molar equivalents.

In one embodiment of the present invention, the cyano source K₄Fe(CN)₆ used in step-(c) is in the proportion of 1 to 0.4 molar equivalents with respect to compound of formula (VII-A); preferably 0.8 to 0.6 molar equivalents.

In one embodiment of the present invention, the ligand imidazole used in step-(c) is in the proportion of 0.8 to 0.2 molar equivalents with respect to compound of formula (VII-A); preferably 0.6 to 0.3 molar equivalents.

In one embodiment of the present invention, the amide solvent used in step-(c) is in the proportion of 6 w/w to 1 w/w with respect to compound of formula (VII-A).

In one embodiment the purity of the compound of formula (VII) is more than 98%.

Finally, the compound of Formula VII and a compound of Formula VIII are reacted to obtain the compound of Formula I,

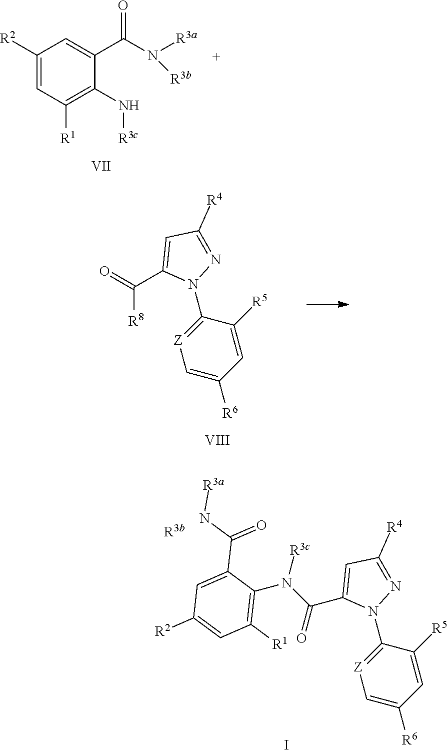

wherein, R¹, R², R^{3a}, R^{3b}, R^{3c}, R⁴, R⁵, R⁶ and Z are as define herein before; R⁸ is OH, Cl, or O—C₁-C₄ alkyl.

The compound of Formula VIII can be obtained by either of the processes disclosed in WO2003015518, WO20030155519, WO2011157664 and WO2013030100.

All or any of the process steps of the present invention may be carried out in continuous, semi-continuous, flow or batch form. Particularly, the process steps of the present invention are carried out in semi-continuous form.

All or any of the process steps may be carried out at a pressure ranging from 0.5 kg/cm² to 250 kg/cm².

Any person skilled in the art knows the best work-up of the reaction mixtures after the end of the respective reactions. In one embodiment, the work-up is usually carried out by isolation of the product by filtration, and optionally washing with a solvent, further optionally drying of the product if required.

The work-up/isolation of the reaction product can also be carried out by a technique which includes but is not limited to decantation, centrifugation, evaporation, ultrafiltration, liquid-liquid extraction, distillation, recrystallization, chromatography, and the like.

The method steps according to the invention are generally carried out under atmospheric pressure.

Alternatively, however, it is also possible to work under reduced pressure or under pressure.

Without further elaboration, it is believed that any person skilled in the art who is using the preceding description can utilize the present invention to its fullest extent. The following examples are therefore to be interpreted as merely illustrative and not limiting the disclosure in any way whatsoever.

The present inventions shall now be described in light of the following non-limiting examples.

### Example 1

### Step A: Preparation of 2-(hydroxyimino)-N-(o-tolyl)acetamide

To a solution of o-toluidine (75 g, 700 mmol) in water (170 mL), hydrochloric acid (73 g, 700 mmol, 35% w/w) was slowly added, followed by the addition of a solution of anhydrous sodium sulphate (636 g, 4478 mmol) in water (800 mL). The resulting reaction mixture was heated to 55° C. Then an aqueous solution of hydroxylamine hydrochloride (73 g, 1050 mmol) in water (280 mL) was slowly added, followed by the addition of chloral hydrate (125 g, 757 mmol) in water (270 mL). The reaction mixture was maintained at 55° C. for 12 h. After completion of the reaction, the reaction mixture was cooled to 20° C. and stirred for 1 h. The solid product obtained was filtered, washed with water (100 mL) and dried to obtain 2-(hydroxyimino)-N-(o-tolyl)acetamide (95 g, 531 mmol, Yield: 76%).

¹H-NMR (400 MHZ, DMSO-d₆) δ 12.16 (s, 1H), 9.47 (bs, 1H), 7.66 (s, 1H), 7.45-7.47 (d, J=7.8 Hz, 1H), 7.19-7.24 (dd, J=7.4 Hz & 0.6 Hz, 1H), 7.14-7.18 (td, J=7.6 Hz & 1.6 Hz, 1H), 7.08-7.13 (td, J=7.4 Hz & 1.3 Hz, 1H), 2.22 (s, 3H); MS: m/z=179.05 [M+H].

### Step B-1: Preparation of 7-methylindoline-2,3-dione

2-(Hydroxyimino)-N-(o-tolyl)acetamide (92 g, 485 mmol) was added lot wise to a solution of sulphuric acid (333 g, 3397 mmol) at 0-5° C. The temperature was slowly allowed to raise to 30° C., and the reaction mixture was stirred for 12 h. After completion of the reaction, the reaction mixture was slowly poured into water (1800 mL). The precipitated solid product was filtered, washed with water (200 mL) and dried to obtain 7-methylindoline-2,3-dione (71.5 g, 485 mmol, Yield: 91%).

¹H-NMR (400 MHZ, DMSO-d₆) δ 11.08 (s, 1H), 7.42 (d, J=7.6 Hz, 1H), 7.31 (d, J=7.6 Hz, 1H), 6.96 (t, J=7.6 Hz, 1H), 2.17 (s, 3H); MS: m/z=162.00 [M+H].

### Step B-2: Preparation of 7-methylindoline-2,3-dione

2-(Hydroxyimino)-N-(o-tolyl)acetamide (10 g, 15.8 mmol) was added lot wise to a solution of sulphuric acid (36.2 g, 369 mmol) and 1,2-dichloroethane (50 mL) at 0-5° C. The temperature was allowed to rise to 30° C., and the reaction mixture was stirred for 12 h. After completion of the reaction, the reaction mixture was slowly poured into water (190 mL). The dichloroethane was removed under reduced pressure, and the remaining suspended solid product was filtered, washed with water (20 mL) and dried to obtain 7-methylindoline-2,3-dione (8.1 g, 50 mmol, Yield: 95%).

### Step C-1: Preparation of 6-chloro-8-methyl-2H-benzo[d][1,3]oxazine-2,4(1H)-dione

7-Methylindoline-2,3-dione (50 g, 261 mmol) and acetic acid (421 g) were mixed at 25° C. to obtain a suspension. To this suspension, hydrogen peroxide (163 g, 1437 mmol, 30% w/w) was added slowly at 25° C. over 15 min under stirring, followed by the addition of conc. hydrochloric acid (82.0 g, 653 mmol, 29% w/w) at a temperature between 3° and 40° C. over 45 min. The reaction mixture was stirred for 3 h at 40° C. The reaction mass was then heated at 70° C. for 4 h. After completion of the reaction, the reaction mass was cooled to 25° C. and then poured slowly onto crushed ice-water mixture (415 g), under stirring. The solid that was obtained by this procedure was filtered, washed with cold water (100 mL) and dried under reduced pressure to obtain crude 6-chloro-8-methyl-2H-benzo[d][1,3]oxazine-2,4(1H)-dione (46 g, 261 mmol, Yield: 83%).

### Step C-2: Preparation of 6-chloro-8-methyl-2H-benzo[d][1,3]oxazine-2,4(1H)-dione

7-Methylindoline-2,3-dione (20 g, 116 mmol) and acetic acid (187 g, 3127 mmol) were mixed at 25° C. to obtain a suspension. To this suspension, hydrogen peroxide (Lot-1) (18 g, 232 mmol) was added slowly over 5 min at the same temperature, followed by the addition of hydrochloric acid (51.0 g, 406 mmol) over 30 min at 15° C. The resulting reaction mass was stirred for 2 h at 25° C. Then hydrogen peroxide (Lot-2) (27 g, 348 mmol) was slowly added over 30 min at 15° C. under stirring. The reaction mixture was heated to 60° C. and stirred for 6 h. Hydrogen peroxide (Lot-3) (12 g, 174 mmol) was added slowly at 20° C. under stirring. The reaction mixture was heated to 60° C. and stirred further for 2 h. The reaction was slowly quenched by pouring it into chilled water (930 g) at 0° C. The resulting mixture was stirred for 1 h at 0° C. The solid obtained was filtered, washed with chilled water and dried under reduced pressure to obtain 6-chloro-8-methyl-2H-benzo[d][1,3]oxazine-2,4(1H)-dione (15.4 g, 72.8 mmol, Yield: 63%).

¹H-NMR (400 MHZ, DMSO-d₆) δ 11.17 (s, 1H), 7.73 (dd, J=2.4 Hz & 0.5 Hz, 1H), 7.69 (dd, J=2.4 Hz & 0.7 Hz, 1H), 2.34 (s, 3H). MS: m/z=209.90 [M−H].

### Step C-3: Preparation of 6-chloro-8-methyl-2H-benzo[d][1,3]oxazine-2,4(1H)-dione

7-Methylindoline-2,3-dione (50 g, 298 mmol) and acetic acid (483 g, 8047 mmol) were mixed at 5° C. to obtain a suspension. Hydrogen peroxide (Lot-1) (69 g, 894 mmol) was added under stirring to this suspension at 5° C. over 15 min. Then hydrochloric acid (Lot-1) (56 g, 447 mmol) was added slowly under stirring at 5° C. for 60 min. The resulting reaction mass was stirred further for 2.5 h at 10° C., then allowed to warm up to 20° C. and stirred further for 23 h. Hydrogen peroxide (Lot-2) (26 g, 313 mmol) was added at 10° C. for 15 min under stirring, and then HCl (Lot-2) (18 g, 158 mmol) was added under stirring slowly over a period of 30 min at 10° C. The reaction mass was stirred at 40° C. for 3 h. The solid obtained was washed with water (600 mL) and dried under reduced pressure to obtain 6-chloro-8-methyl-2H-benzo[d][1,3]oxazine-2,4(1H)-dione (52.70 g, 298 mmol, Yield: 84%).

### Step C-4: Preparation of 6-bromo-8-methyl-2H-benzo[d][1,3]oxazine-2,4(1H)-dione

To a suspension of 7-methylindoline-2,3-dione (10 g, 60.0 mmol) and acetic acid (100 g), hydrogen peroxide (27 g, 360 mmol) was added at 25-30° C. To this reaction mixture, hydrobromic acid (11 g, 57.1 mmol) was added at a temperature between 15-25° C. in 1 h. After stirring for 2 h at 25-30° C., sulphuric acid (0.3 g, 3.0 mmol) was added and the reaction mass was heated to 45-50° C. and stirred further for 8 h. The temperature was slowly raised to 70-75° C. and stirred further for 2 h. After completion of the reaction, the reaction mixture was cooled to 25° C. and poured slowly over crushed ice-water mixture (500 g) under constant stirring at 0-5° C. The solid obtained was filtered, washed with cold water and dried under reduced pressure to obtain crude 6-bromo-8-methyl-2H-benzo[d][1,3]oxazine-2,4(1H)-dione (11.5 g, 45 mmol, Yield: 75%).

¹H-NMR (400 MHZ, DMSO-d₆) δ 11.16 (s, 1H), 7.83 (d, J=2.4 Hz, 1H), 7.78 (d, J=2.4 Hz, 1H), 2.31 (s, 3H)

LCMS: m/z=254 [M−2H].

### Step D-1: Preparation of 2-amino-5-chloro-N,3-dimethylbenzamide

A suspension of 6-chloro-8-methyl-2H-benzo[d][1,3]oxazine-2,4(1H)-dione (17 g, 80 mmol), acetic acid (10 g, 160 mmol) and ethyl acetate (200 mL) was cooled under stirring to 0° C. Methylamine gas was bubbled through this stirred suspension at 0° C. for 15 min (pH=9 to 10). The resulting reaction mixture was then allowed to warm to 25° C. and stirred further for 3 h. After completion of the reaction, the reaction mixture was poured into water (200 g) and extracted with ethyl acetate (200 g). The ethyl acetate layer was dried over anhydrous sodium sulphate, filtered and concentrated under reduced pressure to obtain crude 2-amino-5-chloro-N,3-dimethylbenzamide (12.0 g, 60.4 mmol, Yield: 75%).

¹H-NMR (400 MHZ, DMSO-d₆) δ 8.31 (d, J=4.2 Hz, 1H), 7.37-7.38 (d, J=2.4 Hz, 1H), 7.10-7.12 (d, J=2.4 Hz, 1H), 6.35 (s, 2H), 2.72 (d, J=4.4 Hz, 3H), 2.08 (s, 3H).

MS: m/z=199.00 [M+H].

### Step D-2: Preparation of 2-amino-5-chloro-N,3-dimethylbenzamide

A mixture of 6-chloro-8-methyl-2H-benzo[d][1,3]oxazine-2,4(1H)-dione (0.5 g, 2.4 mmol), methylamine hydrochloride (0.32 g, 4.7 mmol) and potassium carbonate (0.33 g, 2.4 mmol) in ethyl alcohol (10 mL) was stirred for 0.5 h at 25° C., and then heated to 80° C. for 5 h. After completion of the reaction, the reaction mixture was poured into ice-water (50 mL). The precipitate obtained was filtered and washed with water (5 mL). The mother liquor was extracted with dichloromethane (50 mL). The combined dichloromethane layers were dried over anhydrous sodium sulphate, filtered and concentrated under reduced pressure to get a solid. Both the solids were combined to obtain crude 2-amino-5-chloro-N,3-dimethylbenzamide (0.3 g, 1.5 mmol, Yield: 64%).

### Step D-3: Preparation of 2-amino-5-chloro-N,3-dimethylbenzamide

A mixture of 6-chloro-8-methyl-2H-benzo[d][1,3]oxazine-2,4(1H)-dione (0.5 g, 2.4 mmol), methylamine hydrochloride (0.32 g, 4.7 mmol) and pyridine (0.4 ml, 4.7 mmol) in ethyl alcohol (10 mL) was heated at 80° C. for 2 h. After completion of the reaction, the reaction mixture was diluted with water (50 mL) and extracted with ethyl acetate (50 mL). The ethyl acetate layer was dried over anhydrous sodium sulfate, filtered and concentrated under reduced pressure to get a crude solid. The crude solid was triturated with n-hexane (50 mL) to obtain pure 2-amino-5-chloro-N,3-dimethylbenzamide (0.3 g, 1.4 mmol, Yield: 60%).

### Example-2: Preparation of Br-isatoic anhydride

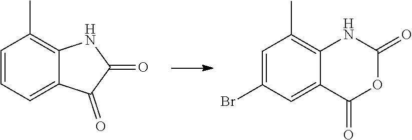

To a solution of isatin (111 g, 1 eq., 623 mmol) in acetic acid (1035 g), 50% aqueous hydrogen peroxide (257 g, 6 eq., 3741 mmol) was added dropwise over a period of 15-20 min at room temperature. The reaction mixture was cool to 12-15° C., followed by the addition of 47% aqueous hydrobromic acid (102 g, 0.95 eq., 592 mmol) over a period of 30 mins. The resulting mixture was warmed to room temperature and allowed to stir for 2 hr. After completion of the reaction, sulfuric acid (3.06 g, 0.05 e., 31.2 mmol) was added to the reaction mixture and heated further at 50±5° C. for 30 min. The temperature of the reaction was then increased to 65±5° C. and stirred further for 5-6 hr at the same temperature. After completion of the reaction, the reaction mixture was gradually cooled to room temperature and pour into ice-cold water (1000 g). The precipitate obtained was filtered through and dried to obtain bromo isatoic anhydride as an orange coloured amorphous solid (148.9 g; Yield: 76.87%)

¹H NMR (DMSO-d₆) δ: 11.16 (br s, 1H), 7.82 (d, J=1.6 Hz, 1H), 7.77 (d, J=1.6 Hz, 1H), 2.39 (s, 3H).

¹³C-NMR (DMSO-d₆) δ: 158.9, 146.9, 139.7, 139.1, 128.6, 127.5, 114.3, 114.1, 112.3, 16.8

### Example-3: Preparation of 2-amino-5-bromo-N,3-dimethylbenzamide

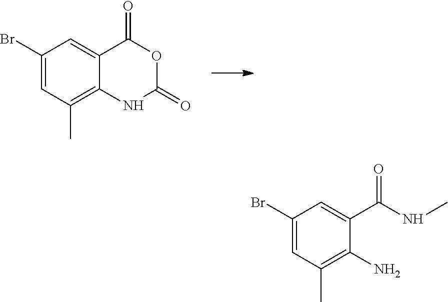

To a stirred solution of Br-isatoic anhydride (1.0 eq., 195 mmol, 50 g) in ethyl acetate (450 g) and acetic acid (1.5 eq., 293 mmol, 17.5 g), methyl amine (1.5 eq., 293 mmol, 23.33 g g, 39% in aqueous solution) was added slowly at 25±5° C. over a period of 15-20 mins. The resulting reaction mixture was continued to stir for 4-5 hr at ambient temperature. After completion of the reaction, an aqueous solution of sodium bicarbonate (13.7 g in 500 g water) was added to the reaction mixture at room temperature and heated to 45±5° C. under stirring for 1 hr. The reaction mixture was cooled to 30±5° C., the organic layer was separated. The aqueous layer was washed with EtOAc (2×45 g). The combined organic layers were washed with saturated (25%) brine solution (45 g), dried over anhydrous sodium sulphate, concentrated under reduced pressure to give crude product (46 g, Yield: 98.57%)

¹H NMR (DMSO-d6, 400 MHz) δ: 8.32 (br d, 1H), 7.49 (d, 1H), 7.22 (d, 1H), 6.35 (br s, 2H), 2.70 (d, 3H), 2.08 (s, 3H);

¹³C NMR (DMSO-d6, 100 MHZ) δ: 168.4, 146.8, 134.2, 127.7, 125.9, 116.1, 105.0, 26.0, 17.2;

### Example-4: Synthesis of 2-amino-5-cyano-N,3-dimethylbenzamide

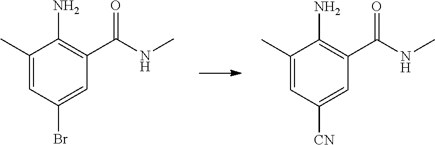

### Condition 1:

To a mixture of n-butanol (100 mL) and N-methyl-2-pyrrolidone (NMP, 25 mL), 2-amino-5-bromo-N,3-dimethylbenzamide (25 g, 102 mmol, 1 equivalent), potassium iodide (0.85 g, 5.1 mmol, 0.05 equivalent), copper(I) iodide (0.98 g, 5.1 mmol, 0.05 eq), potassium ferrocyanide trihydrate ((K₄Fe(CN)₆·3H₂O), 17.39 g, 41.1 mmol, 0.4 equivalent), and sym-dimethylethylenediamine technical ((DMEDA), 4.53 g, 51 mmol, 0.5 equivalent) were added. The reaction mixture was stirred under nitrogen atmosphere at 120° C. for 20 h. After completion of the reaction, the reaction mixture was cooled to 45° C. The solid obtained was filtered, washed with n-butanol, and dried to obtain 14.60 g of 2-amino-5-cyano-N,3-dimethylbenzamide.

¹H-NMR (400 MHZ, DMSO-d₆) δ 8.41 (bs, 1H), 7.80 (d, J=1.6 Hz, 1H), 7.42 (d, J=1.6 Hz, 1H), 7.16 (bs, 1H), 2.72 (d, J=4.8 Hz, 3H), 2.08 (s, 3H) MS: m/z 190.3 [M−1]+

### Condition 2:

To a mixture of N-methyl-2-pyrrolidone (NMP, 77 g), 2-amino-5-bromo-N,3-dimethylbenzamide (50 g, 98.3 assay, 204 mmol, 1 equivalent), potassium ferrocyanide trihydrate ((K₄Fe(CN)₆·3H₂O), 25.9 g, 60.7 mmol, 0.3 equivalent), copper(I) iodide (1.556 g, 8.08 mmol, 0.04 eq), and imidazole (5.56 g, 81 mmol, 0.4 equivalent) were added. The reaction mixture was stirred under nitrogen atmosphere at 160-165° C. for 30 h. After completion of the reaction, the reaction was worked up to afford 2-amino-5-cyano-N,3-dimethylbenzamide (37.7 g, 98.56% yield) as a light brown solid.

¹H-NMR (400 MHZ, DMSO-d6) δ 8.41 (bs, 1H), 7.80 (d, J=1.6 Hz, 1H), 7.42 (d, J=1.6 Hz, 1H), 7.16 (bs, 1H), 2.72 (d, J=4.8 Hz, 3H), 2.08 (s, 3H) MS: m/z 190.3 [M−1]+

### Condition 3:

To a mixture of N,N-Dimethylacetamide (DMAc, 30 mL), 2-amino-5-bromo-N,3-dimethylbenzamide (30 g, 123 mmol, 1 equivalent), potassium ferrocyanide trihydrate ((K₄Fe(CN)₆·3H₂O), 31.27 g, 74 mmol, 0.6 equivalent), copper(I) iodide (2.35 g, 12.34 mmol, 0.1 eq), and imidazole (4.2 g, 61.7 mmol, 0.5 equivalent) were added. The reaction mixture was stirred under nitrogen atmosphere at 145-150° C. for 43 h. After completion of the reaction, the reaction was worked up to afford 2-amino-5-cyano-N,3-dimethylbenzamide (19.3 g, 82.65% yield) as a light brown solid.

¹H-NMR (400 MHZ, DMSO-d6) δ 8.41 (bs, 1H), 7.80 (d, J=1.6 Hz, 1H), 7.42 (d, J=1.6 Hz, 1H), 7.16 (bs, 1H), 2.72 (d, J=4.8 Hz, 3H), 2.08 (s, 3H) MS: m/z 190.3 [M−1]+

### Example-5: Preparation of 1-(3-chloropyridin-2-yl)-N-(4-cyano-2-methyl-6-(methylcarbamoyl)phenyl)-3-((5-(trifluoromethyl)-2H-tetrazol-2-yl)methyl)-1H-pyrazole-5-carboxamide

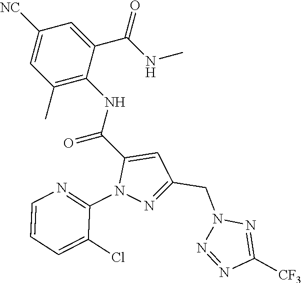 [SMILES: CNC(=O)c1cc(C#N)cc(C)c1NC(=O)c1cc(Cn2nnc(C(F)(F)F)n2)nn1-c1ncccc1Cl]

The above compound is prepared according the procedure mentioned in the example 1 and step 5 of WO2019224678 according to following scheme:

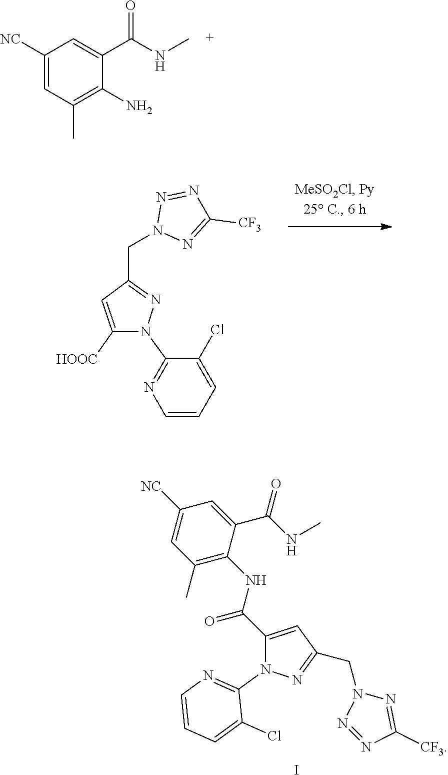

We claim:

1. A process for preparing a compound of Formula VII, 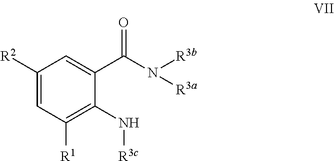
wherein,
R¹ is CH₃, Br or Cl;
R² is CN;
R^{3a} and R^{3b} are independently H, C₁-C₄ alkyl or C₃-C₆ cycloalkyl-C₁-C₄ alkyl; and
R^{3c} is independently H or C₁-C₄ alkyl;
comprising the steps of
a) oxidizing and halogenating the dione of Formula II simultaneously into isatoic anhydride of Formula V using at least one oxidizing reagent and at least one halogenating reagent optionally in the presence of at least one suitable acid; 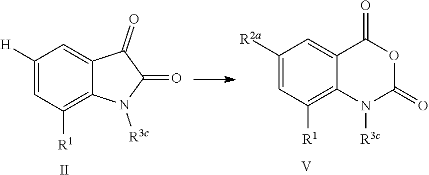
wherein, R^{2a} is F, Cl, Br, or I; R¹ and R^{3c} are as defined herein before,
b) reacting the compound of Formula V with an amine of Formula VI to obtain a compound of Formula VIIA in the presence of at least one suitable reagent; 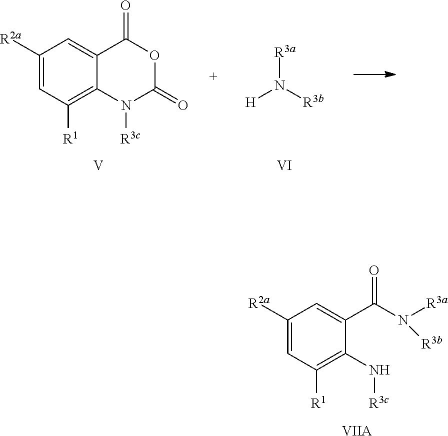
wherein, R^{2a} is F, Cl, Br, or I; R¹, R^{3a}, R^{3b} and R^{3c} are as defined herein before, and
c) reacting the compound of Formula VIIA with cyano source to obtain a compound of Formula VII in the presence of amide solvent; 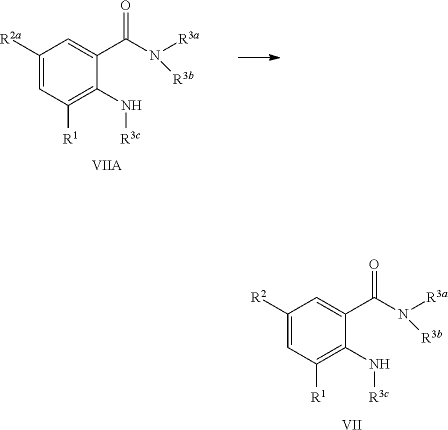
wherein, R^{2a} is F, Cl, Br, or I; R¹, R², R^{3a}, R^{3b} and R^{3c} are as defined herein before.

2. The process as claimed in claim 1, wherein step c is carried out in the presence of ligand and/or catalyst.

3. The process as claimed in claim 1, wherein the cyano source in step-c is metal cyanides which is selected from sodium cyanide (NaCN), potassium cyanide (KCN), copper(I) cyanide (CuCN), zinc(II) cyanide (Zn(CN)₂), sodium ferrocyanide (Na₄Fe(CN)₆), Potassium hexacyanoferrate (III) (K₃[Fe(CN)₆]), or Potassium hexacyanoferrate (II) (K₄[Fe(CN)₆]).

4. The process as claimed in claim 3, wherein the metal cyanides is Potassium hexacyanoferrate (II) (K₄[Fe(CN)₆]).

5. The process as claimed in claim 2, wherein the ligand is selected from 1,2-dimethylethylenediamine (DMEDA), imidazole, 1-butyl-1H-imidazole, 2-picoline, 3-picoline, 2,6-lutidine, 1-methyl pyrolidine, or pyrolidine.

6. The process as claimed in claim 2, wherein the ligand is imidazole.

7. The process as claimed in claim 2, wherein the catalyst is a copper salt and selected from copper (I) iodide (CuI), copper(I) chloride (CuCl), copper(I) bromide (CuBr).

8. The process as claimed in claim 7, wherein the catalyst is a copper salt is copper(I) iodide (CuI).

9. The process as claimed in claim 1, wherein step c is carried out in the presence of amide solvent selected from N,N-dimethylformamide, N,N-dimethylacetamide, or N-methyl-2-pyrrolidone.

10. A process for preparing anthranilic diamide of Formula I, 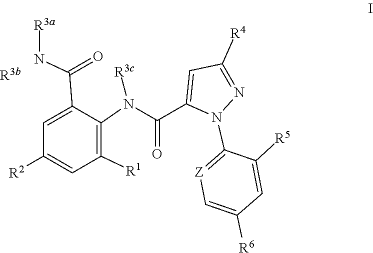
wherein,
R¹ is CH₃, Br or Cl;
R² is CN;
R^{3a} and R^{3b} are independently H, C₁-C₄ alkyl or C₃-C₆ cycloalkyl-C₁-C₄ alkyl;
R^{3c} is H or C₁-C₄ alkyl;
R⁴ is Cl, Br, CF₃, OCF₂H, OCH₂CF₃, 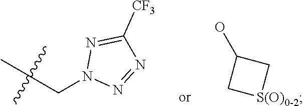
R⁵ is F, Cl or Br;
R⁶ is H, F or Cl;
Z is CR⁷ or N; and
R⁷ is H, F, Cl or Br;
said process comprising the steps of:
a) oxidizing and halogenating the dione of Formula II simultaneously into an isatoic anhydride of Formula V using at least one oxidizing reagent and at least one halogenating reagent optionally in the presence of at least one suitable acid; 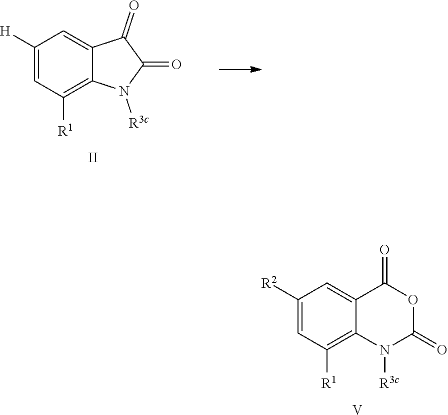
wherein, R¹, R² and R^{3c} are as defined herein before,
b) reacting the compound of Formula V with an amine of Formula VI to obtain a compound of Formula VII in the presence of at least one suitable reagent; 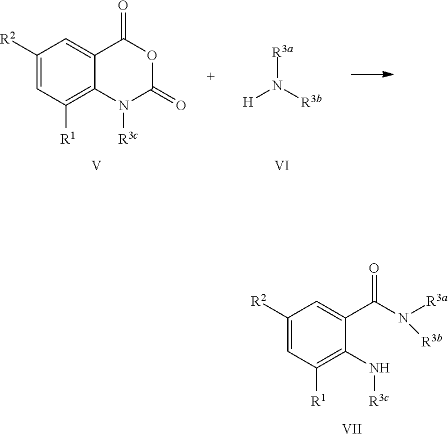
wherein, R¹, R², R^{3a}, R^{3b} and R^{3c} are as defined herein before, and
c) reacting the compound of Formula VIIA with cyano source to obtain a compound of Formula VII in the presence of suitable solvent, optionally in the presence of ligand and catalyst, 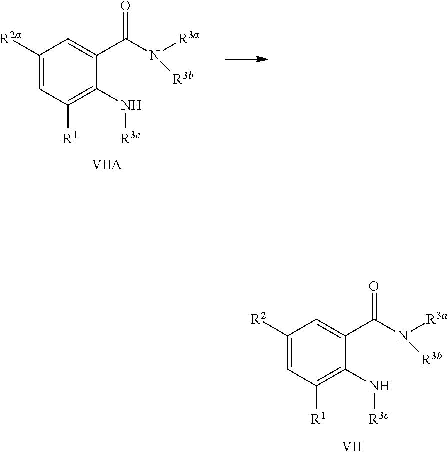
wherein, R^{2a} is F, Cl, Br, or I; R¹, R², R^{3a}, R^{3b} and R^{3c} are as defined herein before
d) reacting the compound of Formula VII and a compound of Formula VIII to the compound of Formula I in the presence of at least one suitable reagent; 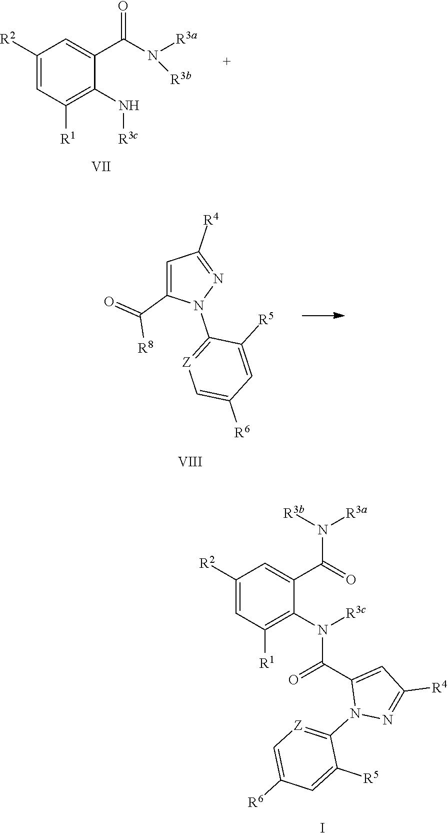
wherein, R⁸ is OH, Cl, X or O—C₁-C₄ alkyl; R¹, R², R^{3a}, R^{3b}, R^{3c}, R⁴, R⁵, R⁶ and Z are as defined herein before, and
wherein, the compound of Formula V may or may not be isolated.

11. The process as claimed in claim 1, wherein the process step (a) is carried out
i) using a suitable halogenating reagent selected from the group consisting of HX, NaX, KX, CuX₂, MgX₂, CsX, ZnX₂, SOCl₂, SO₂Cl₂, COCl₂, X₂, C(═O)(OCl₃)₂, t-BuOCl, NaOCl, chloramine-T, N-halosuccinamides, POX₃, PX₃, PX₅ or metal halides; wherein X is Cl, Br, I or F, particularly, Cl;
ii) using at least one suitable oxidizing reagent hydrogen peroxide, t-butyl-hydroperoxide, tungstic peroxide, m-chloroperbenzoic acid, benzoyl peroxide, hypohalous acid, ceric ammonium nitrate, hypoceric ammonium nitrate, oxone, periodic acid, hydrogen peroxide urea-adduct, sodium perborate, pyridinium chlorochromate and dimethyl sulfoxide;
iii) using at least one acid selected from the group consisting of formic acid, acetic acid, triflic acid, benzoic acid, m-chlorobenzoic acid, butyric acid, propionic acid, glycolic acid, trifluoroacetic acid, para-toluene sulfonic acid, methane sulfonic acid, butyric acid, citric acid, oxalic acid, malonic acid, maleic acid, gallic acid, tartaric acid, ascorbic acid, hydrochloric acid, hydroiodic acid, sulphuric acid, nitric acid, phosphoric acid, and perchloric acid;
iv) at a temperature ranging from 0° C. to 150° C.; and
v) using at least one solvent selected from the group consisting of hexane, heptane, octane, nonane, decane, dodecane, cyclopropane, cyclobutane, cyclopentane, cyclohexane, cycloheptane, cyclooctane, toluene, xylene, mesitylene, benzene, diisopropyl ether, t-butyl methyl ether, tetrahydrofuran, 2-methyl tetrahydrofuran, dioxane, monoglyme, diglyme, methoxy-methane, methoxy-ethane, ethoxy-ethane, di-methoxyethane, di-ethoxyethane, dichloromethane, chloroform, dichloroethane, N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, 1,3-dimethyl-2-imidazolidinone, acetic acid, hydrochloric acid, hydrobromic acid, sulfuric acid and water.

12. The process as claimed in claim 1, wherein the process step (a) is carried out by mixing i) a mixture of the halogenating reagent and the oxidizing reagent, and ii) a mixture of the dione of Formula II and the solvent, at a temperature ranging from 10 to 50° C. and then by heating at a temperature ranging from 15-150° C.

13. The process as claimed in claim 1, wherein the process step (a) is carried out is carried out by adding i) the oxidizing reagent, and ii) the halogenating reagent separately in either sequence in portions or all at once to iii) a mixture of the dione of Formula II and the solvent, at a temperature ranging from 10 to 50° C., followed by heating at a temperature ranging from 15-150° C.

14. The process as claimed in claim 1, wherein the process step (b) is carried out
i) using at least one suitable reagent selected from the group consisting of formic acid, acetic acid, triflic acid, benzoic acid, m-chlorobenzoic acid, butyric acid, propionic acid, glycolic acid, trifluoroacetic acid, para-toluene sulfonic acid, methane sulfonic acid, butyric acid, citric acid, oxalic acid, malonic acid, maleic acid, gallic acid, tartaric acid, ascorbic acid, hydrochloric acid, hydroiodic acid, sulphuric acid, nitric acid, phosphoric acid, perchloric acid, boronic acids, amberlysts, aluminum chloride, zinc chloride, boron trifluoro ether, zinc oxide, titanium tetrachloride, tin chloride, sodium carbonate, potassium carbonate, sodium bicarbonate, potassium bicarbonate, pyridine, N-methyl-2-pyrrolidone, and N,N-dimethylmethanamide;
ii) at a temperature ranging from 0° C. to 150° C.; and
iii) using at least one solvent selected from the group consisting of formic acid, acetic acid, triflic acid, butyric acid, propionic acid, benzoic acid, m-chlorobenzoic acid, carbonic acid, glycolic acid, and trifluoroacetic acid optionally in combination with at least one additional solvent selected from the group consisting of hexane, heptane, octane, nonane, decane, dodecane, cyclopropane, cyclobutane, cyclopentane, cyclohexane, cycloheptane, cyclooctane, toluene, xylene, mesitylene, benzene, diisopropyl ether, t-butyl methyl ether, tetrahydrofuran, 2-methyl tetrahydrofuran, dioxane, monoglyme, diglyme, methoxy-methane, methoxy-ethane, ethoxy-ethane, di-methoxyethane, di-ethoxyethane, dichloromethane, chloroform, dichloroethane, N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, 1,3-dimethyl-2-imidazolidinone and water.

15. The process for preparing anthranilic diamide of Formula I as claimed in claim 10, 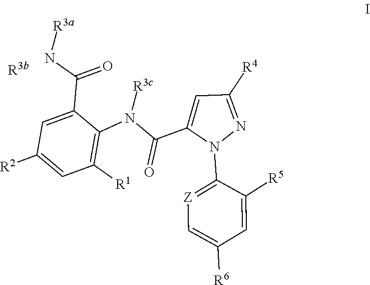
wherein,
R¹ is CH₃, Br or Cl;
R² is CN;
R^{3a} is H and R^{3b} is methyl or 1-cyclopropyl ethyl;
R^{3c} is H;
R⁴ is Br, 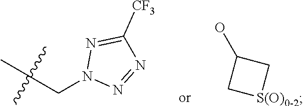
R⁵ is Cl;
R⁶ is H or Cl; and
Z is N.

16. The process as claimed in claim 10, wherein the process step (c) is carried out
i. using metal cyanides which is selected from sodium cyanide (NaCN), potassium cyanide (KCN), copper(I) cyanide (CuCN), zinc(II) cyanide (Zn(CN)2), sodium ferrocyanide (Na4Fe(CN)6), Potassium hexacyanoferrate (III) (K3[Fe(CN)6]), or Potassium hexacyanoferrate (II) (K4[Fe(CN)6]),
ii. using the ligand, which is selected from 1,2-dimethylethylenediamine (DMEDA), imidazole, 1-butyl-1H-imidazole, 2-picoline, 3-picoline, 2,6-lutidine, 1-methyl pyrolidine, or pyrolidine,
iii. using the catalyst, which is a copper salts and is selected from copper(I) iodide (CuI), copper(I) chloride, copper(I) bromide, or copper acetate,
iv. at a temperature ranging from 30° C. to 200° C.; and
v. using at least one solvent selected from the group consisting of heptane, octane, nonane, decane, dodecane, cyclobutane, cyclopentane, cyclohexane, cycloheptane, cyclooctane, toluene, xylenes, mesitylene, benzene, diisopropyl ether, t-butyl methyl ether, tetrahydrofuran, 2-methyl tetrahydrofuran, dioxane, monoglyme, diglyme, methoxy-methane, methoxy-ethane, ethoxy-ethane, di-methoxyethane, di-ethoxyethane, N,N-dimethylmethanamide, dimethyl sulfoxide, N-methyl-2-pyrrolidone, 1,3-dimethyl-3,4,5,6-tetrahydro-2 (1H)-pyrimidinone, hexamethylphosphoramidem, or 1,3-dimethyl-2-imidazolidinone.

17. The process as claimed in claim 10, wherein the step a is carried out using
i) the halogenating reagent is HCl, HBr, SOCl₂, NaOCl, NaOBr, Cl₂ or Br₂;
ii) at least one suitable oxidizing reagent selected from the group consisting of hydrogen peroxide, m-chloroperbenzoic acid, or periodic acid;
iii) at least one acid selected from the group consisting of formic acid, acetic acid, triflic acid, benzoic acid, m-chlorobenzoic acid, trifluoroacetic acid, para-toluene sulfonic acid, methane sulfonic acid, hydrochloric acid, hydroiodic acid, sulphuric acid, orperchloric acid;
iv) at a temperature ranging from 5° C. to 80° C.; and
v) at least one solvent selected from the group consisting of toluene, xylene, t-butyl methyl ether, tetrahydrofuran, 2-methyl tetrahydrofuran, dioxane, monoglyme, diglyme, dichloromethane, chloroform, dichloroethane, N,N-dimethylmethanamide, dimethyl sulfoxide, N-methyl-2-pyrrolidone, acetic acid, hydrochloric acid, hydrobromic acid sulfuric acid or water.

18. The process as claimed in claim 17, wherein i) the oxidizing reagent, and ii) the halogenating reagent are added separately in the sequence in portions to iii) a mixture of the dione of Formula II and the solvent, at a temperature ranging from 10 to 50° C., followed by heating at a temperature ranging from 15-150° C.
